(19) United States
(12) Patent Application Publication
(10) Pub. No.: US 2010/0076060 A1
(43) Pub. Date: Mar. 25, 2010

(54) Aptamers as agonists

(75) Inventors: Bruce A. Sullenger, Durham, NC (US); James McNamara, Iowa City, IA (US); Eli Gilboa, Miami, FL (US)

Correspondence Address: NIXON & VANDERHYE, PC, 901 NORTH GLEBE ROAD, 11TH FLOOR, ARLINGTON, VA, 22203, US

(73) Assignee: DUKE UNIVERSITY, Durham, NC (US)

(21) Appl. No.: 12/385,797

(22) Filed: Apr. 20, 2009

Related U.S. Application Data
(63) Continuation-in-part of application No. 12/066,598, filed on Oct. 2, 2008.
Application No. 12/066,598 is a continuation-in-part of application No. PCT/US07/22357, filed on Oct. 19, 2007.
(60) Provisional application No. 60/716,976, filed on Sep. 15, 2005.
(60) Provisional application No. 60/852,705, filed on Oct. 19, 2006.
(60) Provisional application No. 60/977,589, filed on Oct. 4, 2007.

(51) Int. Cl. A61K 31/7088; C12Q 1/68; C07H 21/04; C12N 5/071; A61P 37/06

(52) U.S. Cl. 514/44 R; 435/6; 536/23.1; 435/325

(57) ABSTRACT

Nucleic acid aptamers are described herein which can transduce a signal into cells by crosslinking a cell surface molecule, thereby inducing of one or more biological activities by the cells; in serving as agonists.

This invention is a continuation-in-part of U.S. patent application Ser. No. 12/066,598, filed 12 Mar. 2008, which was the National Stage entry of International Application No. PCT/US2006/036090, filed 15 Sep. 2006, which claims priority to U.S. Provisional Application No. 60/716,976, filed 15 Sep. 2005. This application is also a continuation-in-part of International Patent Application No. PCT/US2007/022357 filed 19 Oct. 2007, which claims priority to U.S. Provisional Application No. 60/852,705, filed 19 Oct. 2006, and U.S. Provisional Application No. 60/977,589, filed 4 Oct. 2007. The instant application claims the benefit of all of the listed applications, which are hereby incorporated by reference herein in their entireties, including the drawings.

### GRANT STATEMENT

This invention was made, in part, with government support under Grant Nos. 1R01CA104356, HL065222, and 1UL1RR024128 from the National Institutes of Health. Thus, the U.S. Government has certain rights in the invention.

### BACKGROUND

There are many differences between nucleic acid aptamers and antibodies. In addition to the compositional difference (nucleic acid versus glycoprotein), nucleic acid aptamers are typically about one sixth or less the size of antibodies (e.g., 25 kDa versus about 160 kDa). Also, nucleic acid aptamers function in ways more like small molecules than like antibodies, in that nucleic acid aptamers tend to bind grooves and clefts on proteins, and can recognize binding pockets. In contrast, antibodies typically recognize and bind to structure or conformation. Additionally, this type of binding interaction between nucleic acid aptamers and their target proteins makes nucleic acid aptamers well-suited to block interactions between proteins (i.e., function as antagonists).

### SUMMARY OF THE INVENTION

Nucleic acid aptamers are described herein which can transduce a signal into cells by crosslinking a ligand comprising a cell surface molecule, thereby inducing of one or more biological activities by the cells; hence, the nucleic acid aptamers serve as agonists. The invention involves the discovery that despite the physical, compositional, and functional differences between antibodies and aptamers, surprisingly nucleic acid aptamers can be identified which have agonistic activity (“agonist aptamer”). The invention also relates to methods for identifying agonist aptamers, compositions comprising agonist aptamers, and methods of using agonist aptamers.

### BRIEF DESCRIPTION OF THE DRAWINGS

FIG. 1 is a diagram summarizing the SELEX method for aptamer selection.

FIG. 2 is a diagram representing formation of an aptamer dimer agonist from two aptamer monomers using Watson-Crick base pairing.

FIG. 3 is a graph showing days of tumor onset (X axis) plotted against percentage of tumor-free mice (Y axis) showing treatments with dendritic cell transfected with actin RNA, and administered with either: assay negative control aptamer (line with closed stars); assay negative control antibody (line with closed asterisks); aptamer agonist (line with closed diamond); agonist antibody (line with open diamond). Also shown are treatments with dendritic cell transfected with tumor antigen tyrosine-related protein 2 (“TRP-2”) RNA, and administered with either: assay negative control aptamer (line with open squares); assay negative control antibody (line with open circles); aptamer agonist (line with closed squares); agonist antibody (line with closed circles).

### DETAILED DESCRIPTION OF THE INVENTION

Definitions—While the following terms are believed to be well understood by one of ordinary skill in the art of biotechnology, the following definitions are set forth to facilitate explanation of the invention.

“Agonist” and “agonistic” are used interchangeably herein, for purposes of the specification and claims, to describe a molecule which is capable of combining with (binding) a ligand expressed by a cell, and directly or indirectly promoting or inducing (e.g., via stimulating a signaling pathway(s) in a cell leading to) one or more cell activities of a cell. For example, binding of an agonistic molecule to a ligand for the agonist on the surface of a cell triggers one or more signaling pathways in the cell, resulting in inducing one or more cell activities of the cell. An agonist aptamer is a nucleic acid aptamer that functions as an agonist; i.e., comprises agonistic activity, wherein the agonist activity is mediated, at least in part, by the binding specificity of the aptamer to its ligand. This is distinguished from an aptamer onto which is added (as an additional component of the aptamer or incorporated into the aptamer sequence) a CpG motif which motif results in the sole agonistic activity observed, and no agonist activity is mediated from the aptamer binding to its ligand. Thus, an agonist aptamer of the invention lacks a CpG motif. Typically, an aptamer is from about 20 nucleotides to about 80 nucleotides, and often is from about 30 nucleotides to 60 nucleotides in length. The agonist aptamer may comprise modified nucleic acid bases (e.g., modified nucleotides), for example, to improve pharmacokinetics and/or stability (e.g., against nucleases) when administered in vivo. For example, modified purines are know to include, but are not limited to, 2′-O-methyl nucleotides; and modified pyrimidines are known to include, but are not limited to, 2′-deoxy-2′-fluoro nucleotides or 2′-deoxy-2′-fluoroarabino nucleotides. Thus, chemical modifications of nucleotides for agonist aptamers may include, without limitation, phosphorothioate internucleotide linkages, 2′-deoxyribonucleotides, 2′-O-methyl ribonucleotides, 2′-deoxy-2′-fluoro ribonucleotides, 4′-thio ribonucleotides, 2′-O-trifluoromethyl nucleotides, 2′-O-ethyl-trifluoromethoxy nucleotides, 2′-O-difluoromethoxy-ethoxy nucleotides, L-nucleotides, and 5-C-methyl nucleotides.

“Ligand”, used in relation to or for an agonist, is a term used herein, for purposes of the specification and claims, to mean a molecule or receptor expressed on a cell surface and/or as a component of the cell membrane, when bound by an agonist, results in or transmits cell signaling (“signal transduction”) that triggers cell activity. The ligand for the agonist may be any cell surface receptor that is capable of being crosslinked (e.g., oligomerizable or dimerizable) upon binding with the agonist, and thereby transduces a signal into cells. Typically, the ligand for the agonist comprises protein-containing cell membrane molecules comprising glycosylphosphatidylinositil (GPI)-anchored molecules, Tumor Necrosis Factor (TNF) superfamily of receptors (also including TNF Receptor family of receptors), insulin receptor, or a combination thereof; and when two molecules of the ligand are crosslinked on the cell surface by an agonist (having binding specificity for the ligand for the agonist) in the form of a multimer, triggered is one or more cell signaling pathways that initiate one or more cell activities. Ligands for agonists that may be crosslinked resulting in cell signaling and resultant cell activity include, but are not limited to, GPI-anchored molecules implicated in cell signaling are known to include, but are not limited to, CD14, CD16b, CD48, CD52, CD55, CD58, CD59, CD73, CD87, CD90, CD108, CD157, CD160 and Ly-6. Many of these GPI-anchored molecules are known by those skilled in the art to be expressed by cells comprising T cells. Membrane-associated molecules in the TNF superfamily which are implicated in cell signaling are known to include, but are not limited to, CD27, CD30, CD40, CD70, CD95, CD120, CD134 (OX40), CD137 (4-1BB), CD153, CD154, CD178, CD253, CD256, CD257, CD258, CD266, CD268, CD269, CD271. Many of the aforementioned members of the TNF superfamily are molecules known by those skilled in the art to be expressed by cells comprising T cells. Insulin receptor is expressed by cells, such as comprising activated T cells. For ligands for an agonist aptamer, which ligand comprises GPI-anchored molecules or TNF superfamily of receptors, specifically excluded are toll-like receptors. Depending on the ligand for the agonist, a co-stimulation of more than one type of cell surface receptor may be necessary to result in the appropriate cell signaling that results in a cell activity. For example, it is believed that optimal cell activity initiated by crosslinking of CD137 occurs when the cell is co-stimulated (also stimulated; e.g. prior or simultaneously) by crosslinking CD3 on the cell surface. CD134 exemplifies some ligands for agonists which, when crosslinked without co-stimulation (e.g., via the T Cell Receptor), can result in initiation of a cell activity (e.g., T regulatory cells, comprising CD4+, CD25+, CD134+ T cells, capable of suppressing antigen-specific T cell responses), or when crosslinked with co-stimulation (e.g., prior exposure of the cells to an agonist of ligand CD28 or to CD3) can result in initiation of a cell activity (e.g., proliferation of antigen-specific CD8+ T cells, or cytokine production of CD4+ T cells). It is known in the art which of the above-identified ligands for agonists act as a “co-activator receptor” (i.e., works in conjunction with a different cell surface receptor) in co-stimulating the relevant cell type. For example, it is known the art that 24-48 hours following stimulation of T cells with anti-CD3 monoclonal antibody (in generating “activated T cells”), cell surface expression of both CD134 and CD137 was upregulated on CD4+ cells, and CD134 was upregulated on CD8+ T cells (however, co-stimulation with an agonist to CD28 can result in higher levels of cell surface expression of CD134). “Co-stimulation” refers to contacting the cell to be stimulated, for induction of cell activity, with more than one type of agonist (e.g., an agonist to a first ligand (e.g., to CD3), and an agonist to a second ligand (e.g., to OX40 or to 4-1BB), which agonists may be administered simultaneously, or separately but proximate to each other (e.g., within hours of each other, and typically within 12 to 48 hours of each other).

The terms “first” and “second”, as used herein for purposes of the specification and claims, are used to distinguish between two different molecules, or between two different positions on a molecule, as will be more clear from the description.

The terms “cell” or “cells”, as used herein for purposes of the specification and claims, refers to one or more cells or cell types of mammalian origin, and more desirably of human origin. A preferred cell type (preferred cells) may be used in accordance with the present invention to the exclusion of cells other than the preferred cells.

“Cell activity” is used herein, for purposes of the specification and claims, to mean one or more biological activities induced as a result of transduction of cell signaling triggered from binding of an agonist to its ligand. Typically, it is crosslinking of the ligand by the agonist in multimeric form (e.g., dimer, or trimer, etc.) which triggers cell signaling and resultant biological activity. The biological activity may comprise one or more of induction of cell growth, cell survival, cell proliferation, cell differentiation, and cell maturation. Such biological activity can be determined by methods known to those skilled in the art. Specific examples of such biological activities induced (e.g., as compared to a level of the biological activity of cells of the same cell type of which absent is the ligand-agonist binding interaction) are known to include, but are not limited to, cytokine production, apoptosis, cell proliferation, cell differentiation, enhanced cell survival, upregulation of an immune response (e.g., an anti-tumor response, and antiviral response, such as by costimulating a memory CD8+ T cell response to such antigen or enhanced cytotoxic activity), and reversal of cell anergy.

For example, crosslinking of 4-1BB by agonistic antibody on cells, particularly in cells previously co-stimulated with an agonist to CD3, has been reported to have induced activity comprising increased proliferation and cytokine production (e.g., in NK cells), increased cytokine production (e.g., in dendritic cells), enhanced cytotoxic responses (CD8+ T cells), rejection of tumors in vivo (thought to be, in part, as a result of stimulation of NK cells), cytokine production (dendritic cells), proliferation and differentiation of T cells (e.g., CD4+ T cells, CD8+ T cells, intestinal intraepithelial T cells), activation of an antiviral or antitumor response (via activation of CD8+ T cells against viral or tumor antigen; i.e., antigen-specific CD8+ T cells), reversal of T cell anergy, and enhanced T cell survival. Crosslinking of OX40 by antibody on cells, particularly in cells previously co-stimulated with an agonist to CD3 or to CD28, has been reported to have agonist activity in resulting in increased cell proliferation and survival (e.g., in CD4+ T cells), production of cytokines (e.g., CD4+ T cells), enhanced anti-tumor immunity, and induction of cell differentiation (CD4+ T cells).

“Linker” is used herein, for purposes of the specification and claims, to mean a chemical entity that connects a first agonist aptamer to a second agonist aptamer (“first” and “second” used to distinguish between two molecules of the same aptamer or between molecules of different aptamers). The linker may comprise a carbon chain (e.g., from about 5 to about 50 carbons, and which are molecules other than a nucleic acid or protein), nucleic acid molecules, or a combination thereof. Examples of carbon chains as linkers include, but are not limited to, an alkyl, alkene, or aldehyde. The carbon chain may be one or more of substituted, un-substituted, unbranched, or branched. The nucleic acid molecule may comprise one or more of DNA, RNA, single stranded, double stranded, nucleic acid bases found in nature (“natural nucleic acid bases”), or synthetic or modified nucleic acid bases (including, but not limited to, those not found naturally occurring). Typically, the linker comprises a length of from about 3 to about 20 nanometers, and more preferably, from about 5 to about 10 nm. Examples of linkers may include, but are not limited to, carbon chains having a length of from about 10 carbons to about 20 carbons, nucleic acid molecules comprised of between 10 to 40 nucleic acid bases (single-stranded) or base pairs (double-stranded), or a combination thereof. As specific examples of a linker, used here in one embodiment is a linker comprising 18 carbons; in another embodiment, a linker comprising 21 nucleic acid base pairs; and in another embodiment, a linker comprising a combination of a carbon linker (a polyethylene spacer) and nucleic acid bases (e.g., a first nucleic acid molecule on a first end of the polyethylene spacer and a second nucleic acid molecule on a second end of the spacer, wherein the first nucleic acid molecule and the second nucleic acid molecule comprise sequence complementarity to a constant region of an aptamer, and thus are hybridizable to the aptamer under standard conditions known in the art).

“Multimer” is used herein, for purposes of the specification and claims, to mean two or more aptamers are linked together. Thus, a multimer may comprise a dimer, trimer, tetramer, etc. The aptamer molecules in a multimer may be of the same nucleic acid sequence and/or binding specificity, as compared to other aptamer molecules present as part of the multimer. Alternatively, the aptamer molecules in a multimer may be of a different nucleic acid sequence and/or binding specificity as compared to other aptamer molecules present as part of the multimer. For an illustrative example of the latter, a multimer may comprise a dimer of agonist aptamers having binding specificity for 4-1BB (CD137) and a dimer of agonist aptamers having binding specificity for OX40 (CD134), in co-stimulating a cell in synergistically enhancing a cell activity. For example, OX40 and 4-1BB co-stimulation by respective agonist antibodies has been reported to enhance CD830 expansion, resulting in rejection of tumor, and provided significantly enhanced antigen-specific antiviral CD8+ T cell responses as compared to that stimulated by each agonist alone.

“Pharmaceutically acceptable carrier” “is used herein, for purposes of the specification and claims, to mean any compound or composition or carrier medium useful in any one or more of administration, delivery, storage, stability of an agonist aptamer described herein. These carriers are known in the art to include, but are not limited to, water, saline, suitable vehicle (e.g., liposome, microparticle, nanoparticle, emulsion, capsule), buffer, medical parenteral vehicle, excipient, aqueous solution, suspension, solvent, emulsions, detergent, chelating agent, solubililzing agent, diluent, salt, colorant, polymer, hydrogel, surfactant, emulsifier, adjuvant, filler, preservative, stabilizer, oil, and the like as broadly known in the pharmaceutical art.

Presented herein is a more detailed description of the invention. Certain aspects of the invention are described in greater detail in the non-limiting Examples that follows.

### Example 1

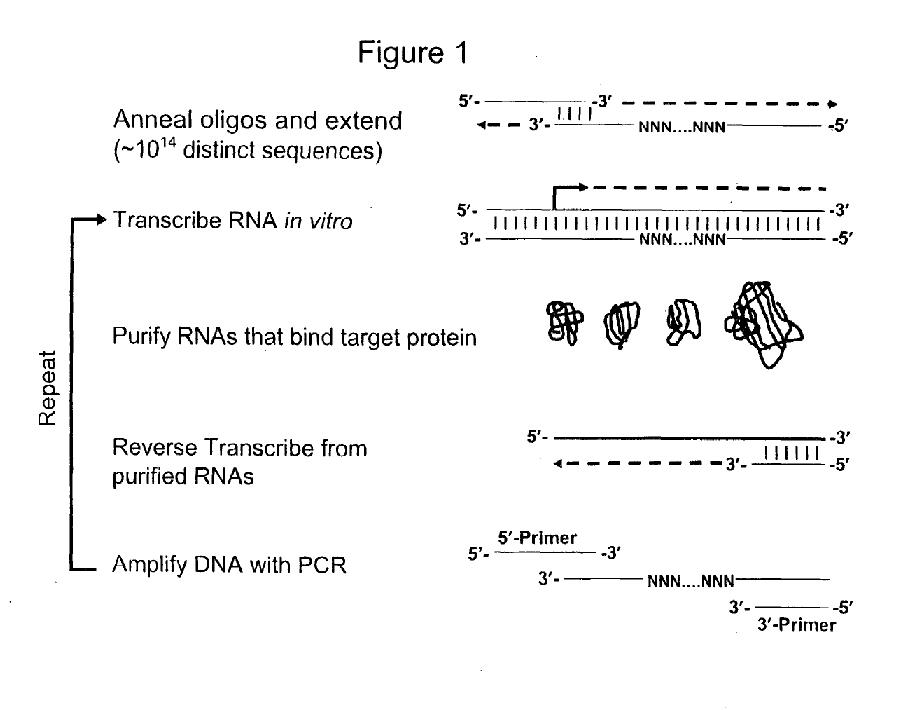

As well known in the art, nucleic acid aptamers can be generated by in vitro screening of complex nucleic-acid based combinatorial shape libraries (e.g., >10¹⁴ shapes per library) employing a process termed SELEX (for Systematic Evolution of Ligands by EXponential. Enrichment). SELEX (FIG. 1) is an iterative process in which a library of randomized pool of RNA sequences is incubated with a selected protein target. Interacting RNA is then partitioned from non-binding RNA and subsequently amplified through reverse transcription followed by amplification via polymerase chain reaction (RT/PCR).

Next, this DNA template is used to create an enriched RNA pool through in vitro transcription with a mutant T7 RNA polymerase that allows for the incorporation of 2′fluoro-modified pyrimidines. These modifications render the RNA more nuclease resistant. The steps leading to the creation of the enriched RNA pool are referred to as a “selection round”. The selection rounds against a protein target are typically continued until a plateau in binding affinity progression had been reached. Individual clones may then be isolated from the pool and sequenced.

### Example 2

Using the general methodology described above, RNA aptamers were selected to cell membrane receptor 4-1BB (CD137). The pyrimidines in the RNA used in these selections were 2′-fluoro-modified in order to protect the RNAs from extracellular RNAses, and thus make them suitable for in vivo studies or as therapeutics. Three processes of selection were carried out for high-affinity RNA aptamers to 4-1BB. Briefly, a “preclearing” protocol was used to remove sequences in the library that are nonspecific binders. For example, prior to rounds of selection using the target 4-1BB, the RNA pool was incubated with human IgG1-bound Protein A-coated polymeric beads in a low salt buffer for about 30 minutes at room temperature. RNA pool not bound to the beads (precleared RNA pool) was then incubated with a 4-1BB-Fc fusion protein bound to the polymeric beads for 30 minutes at 37° C. The beads were then washed with buffer, and the RNA was recoved using a phenol-chloroform-isoamyl alcohol extraction. The selection was typically initiated (early rounds) with incubation in a low salt buffer (e.g., 50 mM), with subsequent rounds being incubated in an increasing salt gradient (e.g., up to 150 mM), so as to increase the stringency of the selection process to select for RNA pool members having higher binding affinity. The affinity of the RNA pool for 4-1BB was measured using a double-filter nitrocellulose assay, as known in the art (see, e.g., Example 3, herein). After the final round of selection, RNA was reverse transcribed and cloned into a DNA vector for sequencing.

The first selection was carried out with a fusion protein of the extracellular portion of mouse 4-1BB and the fixed portion of human IgG1 (Fc) using an RNA library with 40 randomized bases. A total of 12 rounds of selection were completed. The round 12 pool of aptamers bound mouse 4-1BB with a dissociation constant of approximately 50 nM. The second selection was carried out with fusion proteins of the extracellular portions of either mouse 4-1BB, or human 4-1BB, fused with Fc. Six rounds of selection were carried out with the mouse 4-1BB fusion, followed by two rounds with the human 4-1BB fusion, and then four additional rounds alternating each round between mouse 4-1BB and human 4-1BB isoforms. This second selection was also carried out with an RNA library with 40 randomized bases. The pool of aptamers obtained from this selection bound human 4-1BB and mouse 4-1BB with dissociation constants of approximately 23 nM and 200 nM, respectively. The third selection was carried out with the human 4-1BB-Fc fusion with an RNA library containing 20 randomized bases. After 9 rounds, the RNA pool obtained from this library bound human 4-1BB with a dissociation constant of approximately 20 nM. Illustrative examples of the of the nucleic acid aptamers isolated from the aforementioned selections against 4-1BB are shown in Table 1; wherein nucleic acid sequences comprising SEQ ID NOs: 1-46 represent the variable regions from clones with binding specificity to mouse 4-1BB; nucleic acid sequences comprising SEQ ID NOs: 47-75 represent the variable regions from clones with binding specificity to both human 4-1BB and mouse 4-1BB; and a nucleic acid sequence comprising SEQ ID:76 represents the RNA library constant regions, with a constant region (comprising nucleic acid bases in positions 1-22 of SEQ ID NO:76) flanking the 5′ end of the variable regions, and a constant region (comprising nucleic acid bases in positions 63-96 of SEQ ID NO:76) flanking the 3′ end of the variable regions, and the variable regions being designated by N(40) in Table 1 (nucleic acid bases in positions 23-62 of SEQ ID NO:76). It is understood by those skilled in the art that the variable regions may be flanked by constant regions other than those comprising the constant regions depicted in SEQ ID NO: 76, while still retaining binding activity for 4-1BB.

TABLE 1

| SEQ ID | |
|---|---|
| NO: | 5′ to 3′ Nucleic Acid Sequence |
| 1 | CGACCGAACGUGCCCUUCAAAGCCGUUCACUAACCAGUGC |
| 2 | CGACCGAACGUGCCCUUCAAAGCCGUUCACUAACCAGUGG |
| 3 | CGACCGAACGUGCCCUUCAAAGCCGUUCACUAACCAGUGA |
| 4 | GAAGUGACAGCUCCCAGCGCUUCAAAGCUCAUCUAUAACU |
| 5 | CAGAAACUAGACCUCCGAUCGGACACCCGGUCCCUUCGUC |
| 6 | GAAGCACAAUAGGCCGCAACACUUCAAAACCCAUUCAAUC |
| 7 | CAAGCACUCUUCAGGCUAAGGACUCUCUUGACACCCCGC |
| 8 | GCACAGCAACACCACGACCCCCCCUAGGCUUCCGCCCGCC |
| 9 | GCACAGCAACACCACGACCCCCCCUAGGCUUCCGCCCGCG |
| 10 | GCACAGCAACACCACGACCCCCCCUAGGCUUCCGCCCGCA |
| 11 | UAACGGCCCAAUGACUUCGCCUUACUGCCCCCCUAAGCUUC |
| 12 | AAAGCGACAAUUCUUACUACUCCCCAAGCUCCACGCCUUU |
| 13 | AAGACGAUACCUAGCCUCAAAAUUCCUCCCCCGACUUCCU |
| 14 | CGAGAACCCGCAUCUUCGGAUGCGCCCCCCUAGGACUUAC |
| 15 | GACCAAGGGCAGCAUCACCGUUCCCCCCCUAGGAGCUUAC |
| 16 | TAACGGCCCAAUGACUUCGCCUUCUGCCCCCCUAAGCUUC |
| 17 | CGCUCUCUCACAACCACGACCUCCGAUCUGAUAAUUCGUC |
| 18 | GCACCAAACACCGGUUCAGAACCCAUCAUGUAACUCCUUG |
| 19 | AACUACCUCCUCGAACCAUAGUUCAACACCAUCCAGCCAU |
| 20 | CUCCUAGACAGACGAUGUCUGAACGACUCUUGAAGGGCAG |
| 21 | CCGCCAUUAACCCUGAACAGAAUCUUCCUAUCCCAACCCG |
| 22 | UCACCCGACGAUACCUAACUUCCUCCGACAAGCCUUUGCC |
| 23 | GCACAACAUCCUCNGUGANACCACACUUCGCCAANCUCUG |
| 24 | AUCAGUACAUGCUCCGGGGCAGCCGAAUAUCUCGCGCUU |
| 25 | GACCACCACAGCGACAGGCUCGCACGACUCUCGAAGAGG |
| 26 | CAAGCACUCUUCAGGCUAAGGACUCUCUUGACACCCCGC |
| 27 | CCUCCUUCCUAACCCGCAAACACGAUUGGCUCGAAACUUC |
| 28 | AACCGACCGCAGAUCUGGACACUAGCUUUCGCGAGGAUU |
| 29 | GCUUCUACAGGGGGUAAAGAUUCGCUCAUCACUCGCAAAA |
| 30 | CGACCGAACGUGCCCUUCAAAGCCGUUCACUAACCAGUGG |
| 31 | GCACUCUUACUUCUCGCCCACCAACCGAACCCAGCCAUCU |
| 32 | CUGACUGCCACUUCUUCCCAGCAAUCUUCACCGUCCUUCU |
| 33 | AAGUUAAUCGCGCCUUACCAGCUUCAGGACCAUUCAAUUC |
| 34 | CUUCCACACUCUCCUAGCUGGACAACAGACUAACCUUCUU |
| 35 | CACCCGUCUGCCAGACGCUCGACCCAAACUCCAGCCUCU |
| 36 | UGAUCCAAAACCAAAUAGCAGUAGACUCUCGAAGAGGACG |
| 37 | CAAACCCAUGCAAGACUCUUCAAUCUCCAACCCCUUCCAA |
| 38 | CUUCAACAACGACUCGCCAUACAACAACACACAACCCCUU |
| 39 | GAAGACCAAACGAGCCACCUUCAAGUCCCAUCCAACCCCU |
| 40 | CGUCAACCCCUUUCAGUCUCCCCGUAAGCAUCUCUUCCAC |
| 41 | CUCCAAUCCCCGAAAUCUUCAAGCUGCUCUCCAGUCCCCA |
| 42 | ACACUGAAAACGGGCAAGUGACUACGAUUCGUGUCAGAC |
| 43 | CUCCUAGUCAGAGGAUGUCUGAACGACUCUUGAAGGGCAG |
| 44 | UAAGCGGCCUCGAACACCGAUUGACUACUGCCCGAACAUC |
| 45 | CCCCCUCGCAAGACCCUCAUCGCAUCUCAAGACACCUACC |
| 46 | ACGACCACCAACGAUUGAGCAGCUAGAUUCGACCGACACU |
| 47 | UGCGAUCCACGGUCUAUUCACCAGUAACCUCUGACCAAAA |
| 48 | CUCCAGACAUAGUAUCCGCCAGUAACCUCUGUAACGCCCG |
| 49 | CGCACUACCUGCACAUUGACCAGUACCCCUGUGCCACGCU |
| 50 | CCGACGACCUCCUUCACCUCCCCUUCCUCGAACUCCAACC |
| 51 | ACCUGACGCAUACCUCCUCCCACCGCAAGUGCCCUCCGCU |
| 52 | ACGACCUCUUUGAUUACCUGGGCUGAUUUGCCAUCUUCAC |
| 53 | ACGACCUCUUUGAUUACCUGGGCUGAUUCGCCAUCUUCAC |
| 54 | CCUCACUGCGUAAUCCGCCGUGAUUCACCGCUGUACACGA |
| 55 | CACCAUCGAUAUCACGCACUGACACCAGUACCCUUGCGCA |
| 56 | UGCUCCAAUCGGAACCAUCUCGCCGCUCGCUCUCCAUUAC |
| 57 | GAAACCUGUGCGCAUGAAGAGCUGCUAACCCGUUACGAAC |
| 58 | GAUCAAACUAAUCGCCCUCUAACUUGAUCGGCCCGACUCC |
| 59 | CCCCCGCGUCCUCCCAACCCAAAAUUCCCCUGGCACCCUC |
| 60 | UGCUCUUAAUCUCCCUAAGAACCAUCUCCAUAGUCCUCCG |
| 61 | GGAUCGACCCGCUUGCUGCGACACGACCGUCGUAACCAA |
| 62 | UGGAUCUCGCUCUGGCGCUUAGCUCCGCAAAUCUGCCGUU |
| 63 | CACUUCCACUCACAACGGCAAAACUUCAACUCCAAUACCCU |
| 64 | ACCAGCUUCCCUGGGCCUGCUGUGAACUCCAGCCUGACUA |
| 65 | GCCUUUACACCGACCCUGCCAAGAACGAAGCGCGAACAAC |
| 66 | CACCUUCCCUUUCUCCAAGACAAGCCUCAUGUCAAACUCU |
| 67 | GGCUUCCCUGCACUUGAUCUGGCCCGACCACCAGCCUGUG |
| 68 | CGAUCCUCUGUACACGACACUCAUUCCACAGUACGGGACG |
| 69 | CACCCUUCGUCUCAACCACCCAACUCUCCAUGUCCCUAAC |
| 70 | CAAACUUCCCUGUGGUUAUAGCACUGCGUUGGGUCCCUUG |
| 71 | AUACCUGUACCUUGCAACACUGCCCGACGACGCACCCGCA |
| 72 | CCACACGCUACUAUGAACCGCUGCGUUCAAACUUGGGAUC |
| 73 | GACCAGUCUAACCAUCCACACUCUGUCAUUGCCCAGCGCC |
| 74 | ACCUUUACCGCCGACCCGGACAGAGUGAGCCUCUGCAGAG |
| 75 | CUCCAGAACCCUUCGACCUGCACGACGGUCUUCCUCGCGU |
| 76 | GGGAGAGAGGAAGAGGGAUGGGNNNNNNNNNNNNNNNNNNNNNN |
| | NNNNNNNNNNNNNNNNNNCAUAACCCAGAGGUCGAUAGUACUGG |
| | AUCCCCCC |

### Example 3

Using the general methodology described in Examples 1 & 2 herein, RNA aptamers were selected to cell membrane receptor OX40 (CD134). An 80 nucleotide combinatorial RNA library was created which contained a variable region of 40 randomized bases. This library was subjected to two “preclearing” steps to remove RNAs specific to human IgG Fc as well as protein G. The randomized RNA library was incubated with 1 nmol of human IgG1 at 37° C. for 30 minutes. IgG-bound RNA was removed by centrifugation over a 0.4 micron nitrocellulose column. The preclearing step was subsequently followed by incubation with magnetic protein G-coated beads. After bead pelleting through exposure to a magnet, the supernatant was applied to a nitrocellulose column. All binding reactions were carried out in 150 mM NaCl, 2 mM CaCl₂, 20 mM Hepes (pH 7.4), 0.01% BSA buffer.

To enrich for OX40-binding RNAs, murine OX40 human IgG Fc fusion protein was immobilized by coupling to protein G-coated magnetic beads. The bead-coupled OX40 fusion protein was incubated with the precleared RNA pool, and then washed three times with a 20 fold excess volume wash buffer (150 mM NaCl, 2 mM CaCl2, 20 mM Hepes (pH 7.4)) to remove non-interacting RNA. RNA bound to OX40 was extracted by a 30 minute incubation in phenol: chloroform: isoamyl alcohol (25:24:1). The RNA was amplified by reverse transcription followed by PCR. A secondary, enriched RNA pool was created with transcription using a 2′OH purine, 2′F pyrimidine nucleotide mixture using T7 polymerase.

Eleven rounds of selection were performed with increasing stringency throughout the selection process by increasing the RNA: OX40 protein ratio in the selection reaction. Aptamers from rounds 9 and 11 DNA were cloned into a plasmid vector, and single colonies were sequenced and amplified by low cycle PCR amplification following by in vitro transcription. Binding constants for the clones were determined using filter-binding assays in a buffer composed of 20 mM Hepes pH 7.4, 150 mM NaCl, 2 mM CaCl2. To determine the affinities of monomeric aptamers (e.g., which may be expressed as K_{d} or dissociation constant), serial dilutions of murine OX40 IgG Fc fusion protein, human IgG1, or protein G were incubated with monomeric 5′ ³²P radiolabeled aptamers at 2000 cpm/μL. The mixture was passed over a stack of membranes consisting of a nitrocellulose membrane and nylon membrane through application of a vacuum. The membranes were exposed to a phosphoimager screen, scanned and quantitated using a phosphoimager. Finally, differential fractions of RNA bound were calculated and graphed using commercially available software, and affinity was then expressed as K_{d}. The predicted secondary structure of the resultant nucleic acid aptamers was determined by utilizing the algorithm m-fold using default settings for folding parameters. Illustrative examples of the nucleic acid aptamers isolated from the aforementioned selections against OX40 are shown in Table 2; wherein nucleic acid sequences comprising SEQ ID NOs: 77-87 represent the variable regions from clones with binding specificity to OX40; and a nucleic acid sequence comprising SEQ ID:87 represents the RNA library constant regions, with a constant region (e.g., nucleic acid bases in positions 1-41 of SEQ ID NO:76) flanking the 5′ end of the variable regions, a constant region (e.g., nucleic acid bases in positions 82-106 of SEQ ID NO:87) flanking the 3′end of the variable regions, and the variable regions being designated by N(40) in Table 2 (nucleic acid bases in positions 42-81 of SEQ ID NO:87). It is understood by those skilled in the art that the variable regions may be flanked by constant regions other than those comprising the constant regions depicted in SEQ ID NO: 87, while still retaining binding activity for OX40.

TABLE 2

| SEQ ID | |
|---|---|
| NO: | 5′ to 3′ Nucleic Acid Sequence |
| 77 | CAGUCUGCAUCGUAGGAAUCGCCACCGUAUACUUUCCCAC |
| 78 | AUACCAGGAUCACAUCCUGAGGAACCCCGGCUCCCAACCU |
| 79 | CUUUAAUCCUCGCACUCAGCGCGCAUCACCCUUGACAUCA |
| 80 | CAAACCAGCUAUUUCCUGAGGUACCCCGGCUCUCCAUGG |
| 81 | AUACCAGCGAAUAACUCGCUGAGGAACCCGACUCACAAA |
| 82 | CUUUUGAAUGCUCCCUUCACUCCAAGCGGCAGCCUAUCGU |
| 83 | AUACCACCUAGUGUGAGGAACCCCGCUUCCUAGACUGCG |
| 84 | CAACCCAUCCAUACCAACAUGGAGCUAAUGUUAGUCAACG |
| 85 | CACUAACUCCAGCGACUCCAUCGCCAUUCCAACUCUACGC |
| 86 | CCGUCAACCAAUCUCUCGCUUCACACGGCCAUGCCUCCAU |
| 87 | GGGGAAUUCUAAUACGACUCACUAUAGGGAGGACGAUGCGGNNN |
| | NNNNNNNNNNNNNNNNNNNNNNNNNNNNNNNNNNNNNCAGACGA |
| | CUCGCUGAGGAUCCGAGA |

### Example 4

In this example, demonstrated is a method for identifying nucleic acid aptamer, having binding specificity against a cell surface receptor or cell membrane protein-containing molecule, which functions as an agonist by binding a ligand for the agonist; and also provided herein are illustrative examples of agonist aptamers according to the invention.

### Multimerization

In one illustrative example, using the methods illustrated in Example 2 herein yielded several sequences that bind 4-1BB (of human origin, mouse origin, or both human and mouse origins) with high affinity (e.g., K_{d} of less than 50 nm). These high-affinity binders were tested for their ability to stimulate 4-1BB in vitro. Because aptamers generally bind only one molecule of ligand per aptamer molecule, aptamers were multimerized in order to crosslink 4-1BB on the cell surface. To multimerize the aptamers, a biotin-streptavidin was used as a linker. The aptamers were labeled on their 5′-ends with biotin by spiking the aptamer transcriptions with a biotin-labeled nucleotide. Following purification of the biotin-labeled aptamers, they were denatured and then renatured to allow for folding; and then incubated with streptavidin-coated beads. Because each streptavidin protein is able to bind up to four biotin-conjugated molecules, the streptavidin-binding step multimerizes the aptamers on the surface of the beads. Aptamers bound to streptavidin-coated beads were then tested for their ability to function as agonist aptamers by stimulating 4-1BB on mouse T cells.

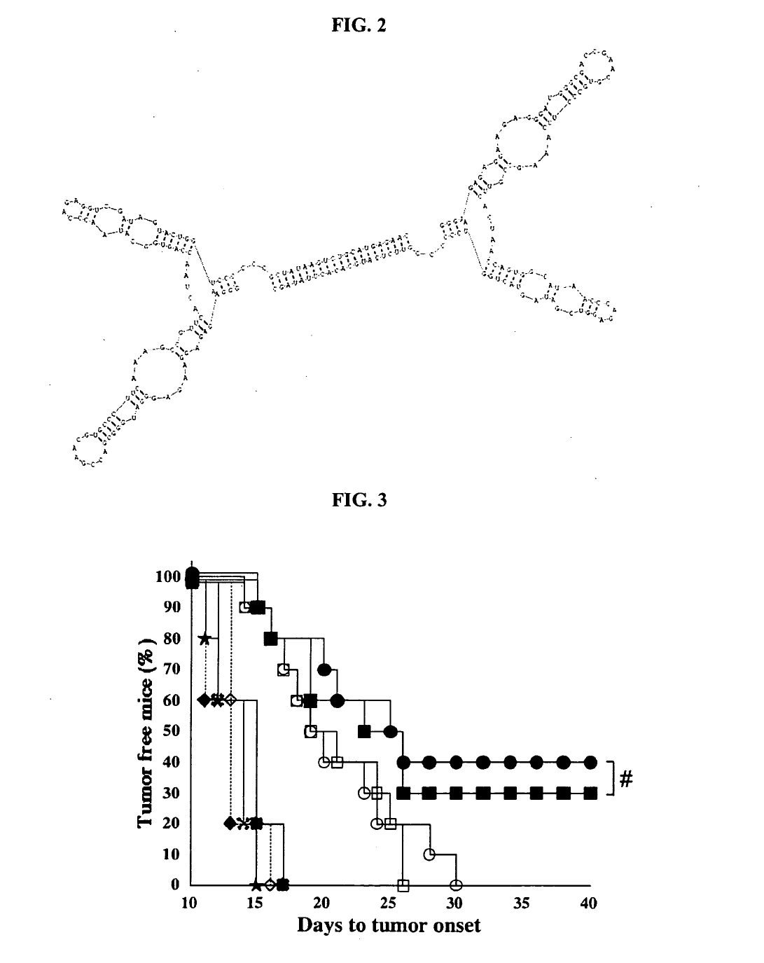

In another illustrative example, aptamers with binding affinity to 4-1BB were multimerized into dimers using a linker comprising nucleic acid bases. For example, an aptamer comprising a nucleic acid sequence of SEQ ID NO: 88 (SEQ ID NO: 76 with SEQ ID NO:3 as the variable region) was synthesized to comprise a first oligonucleotide tail, as shown in SEQ ID NO: 89 (nucleic acid bases in positions 97-117 of SEQ ID NO:89). The same aptamer was also synthesized as a monomer to comprise a second oligonucleotide tail which was complementary and hybridizable to the first oligonucleotide tail, as shown in SEQ ID NO: 90 (nucleic acid bases in positions 97-117 of SEQ ID NO:90). The respective monomers were then combined via hybridization of the complementary oligonucleotide tails by a standard method of denaturation and subsequent annealing (e.g., denaturation at 65° C. followed by annealing at 37° C.). The resultant dimers were then purified. FIG. 2 is an illustration of one such dimer produced in accordance with the present invention (a nucleic acid molecule comprising SEQ ID NO:89 hybridized to a nucleic acid molecule comprising SEQ ID NO:90).

### Methods for Identifying an Agonist Aptamer

A method for identifying a nucleic acid aptamer having agonistic activity comprises:
(a) contacting a nucleic acid aptamer or multimer thereof with cells comprising a ligand (e.g., protein-containing cell membrane molecule present on the cells) to which the nucleic acid aptamer has binding affinity; and
(b) detecting a cell activity (one or more cell activities), which activity is initiated by cell signaling resulting from the binding of the nucleic acid aptamer to the ligand; wherein detection of the cell activity is an indication that the nucleic acid aptamer comprises an agonist aptamer.

In this illustrative example of the method according to the invention, the nucleic acid aptamer is contacted with cells under conditions sufficient for the nucleic acid aptamer to bind to its ligand expressed by the cells, and then the cells are assayed for a cell activity comprising cytokine production (e.g., by detecting cytokines released from the cell treated with the nucleic acid aptamer). CD8+ T cells were isolated from the spleens of BALB/C mice and then incubated in 96-well round-bottomed dishes at 10⁶ cells per well for 20 hours with a monoclonal antibody against CD3 (as a costimulatory signal for T cells, in activating the T cells; (1 μg/ml)). Multimers of an aptamer with binding specificity for 4-1BB, and formed using a linker as described above, were added to the wells containing the co-stimulated T cells. After incubating the cells for an additional 48 hours, an ELISA was carried out to measure relative levels of the cytokine interferon-gamma in the cell supernatants. As a positive control, an anti-4-1BB antibody that is known to stimulate 4-1BB (3H3) was added at 5 μg/ml to some of the wells. As an assay negative control for the antibody, an isotype-matched control antibody (rat IgG2a) was added to other wells at 5 μg/ml. As an assay negative control for the multimers having binding specificity for 4-1BB, multimers were formed using a randomized library of RNA sequences using the linking as described above. These assay negative controls were evaluated in the same assays along side the RNA aptamer multimers having binding specificity for 4-1BB. In detecting for the presence or absence of induction of cell activity following contact with aptamers (in the form of multimers) having binding specificity for 4-1BB, comprising for cytokine production in these cells, the anti-4-1BB agonistic antibody typically produced a 3 to 4-fold increase in interferon-gamma, compared with the isotype-matched control antibody. The multimers comprising randomized RNA library (assay negative control) induced a comparable level of interferon-gamma as the isotype-matched assay negative control antibody. Of the tested multimers of nucleic acid aptamer having binding specificity for 4-1BB, at least two multimers induced substantial increases in the interferon-gamma levels over the assay negative controls, identifying these two multimers as agonist aptamers, and that 4-1BB functions as a ligand for the agonist. One of these RNA aptamer multimers induced interferon-gamma levels that were 2.7- to 3-fold greater than that induced by the multimers comprising randomized RNA library. In some cases, an agonist aptamer comprising a multimer induced interferon-gamma levels that were greater (e.g., 2 to 3 fold) than that induced by agonistic anti-4-1BB antibody. Flow cytometry, using aptamer dimers labeled with fluorochrome AF488, confirmed that the aptamer dimers having binding affinity for 4-1BB were binding to 4-1BB expressed on the cell surface of cells co-stimulated with anti-CD3 antibody.

In another embodiment of detection of a cell activity induced or triggered by agonist aptamer binding to a ligand for the aptamer on the cell surface, a cell activity comprising cellular proliferation was assayed in cultures of mouse CD8+ T cells stimulated in the same manner as described above in the assay for cytokine production. For example, using a multimer as described above in this Example and which also showed initiation of a cell activity comprising cytokine release, detected was a 3-fold increase in cell proliferation in response to contacting this multimer with the cells, and as compared with a relevant assay negative control aptamer. Such proliferation initiated in response to the agonist aptamer binding was comparable to that of the anti-4-1BB agonistic antibody positive control.

In another example of detection of a cell activity induced or triggered by agonist aptamer binding to a ligand for the agonist on the cell surface, a cell activity comprising inhibition of tumor cell growth was assayed. In this regard, administration of agonistic antibodies which bind 4-1BB have been demonstrated to induce regression of mastocytoma tumors (which express cell surface 4-1BB). In an experimental model, P815 mastocytoma tumor cells (about 8×10⁴ cells) were implanted subcutaneously into mice. When tumors developed and reached about 4 to 5 mm in diameter, RNA aptamer dimers having binding specificity for 4-1BB were injected intratumorally (30 μg) and again 48 hours later for a total of 2 injections. As a positive control, some tumors were injected intratumorally with anti-4-1BB agonistic antibody (30 pg, 2 injections). As negative controls in this system, tumors were either injected with dimers of RNA aptamers lacking binding affinity for 4-1BB, or isotype-matched antibody lacking binding affinity for 4-1BB. Injection of aptamer dimers having binding specificity for 4-1BB resulted in inhibition of tumor growth, resulting in regression of most tumors. A similar effect of tumor regression was seen with the positive control (anti-4-1BB agonistic antibody), but no regression was noted in tumors treated with the negative controls. It is important to note that in demonstrating agonistic activity of multimerized RNA aptamers having binding affinity for 4-1BB in this assay and the other assays described above, RNA molecules (lacking detectable binding affinity for 4-1BB) comprising dimers were used as assay negative controls. Thus, the results of these assays confirm that the activity of the nucleic acid aptamers in stimulating a cell activity is a result of agonist activity (i.e., mediated by agonist aptamer binding the ligand for the agonist) rather than by nonspecific effects on the cells mediated by the molecules being RNA in nature.

Thus, demonstrated are (a) a method for identifying an agonist aptamer; (b) agonist aptamers, and particularly comprising multimers of aptamers which can function as agonists; (c) ligands for agonists, which can be bound by agonist aptamer; and (d) examples of cell activities which can be used to detect agonist aptamer function or identification.

### Example 5

This example describes additional embodiments of demonstrating a method for identifying nucleic acid aptamer, having binding specificity for a ligand comprising a cell surface receptor or cell membrane protein-containing molecule on cells, which functions as an agonist by binding a ligand for the agonist; and also provided herein are additional illustrative examples of agonist aptamers according to the invention.

### Multimerization

In this example, nucleic acid aptamers were dimerized to form a multimer, wherein the linker used to join two aptamer monomers was a carbon-based linker. The linker comprised a scaffold comprising a 19 nucleic acid base (DNA nucleotide) sequence at either end of an 18 carbon spacer, the nucleic acid base sequence being complementary to sequence of the constant region at the 3′ end of the RNA aptamer (e.g., for RNA aptamers depicted in Table 2, the linker comprised a sequence of: 5′ TCTCGGATCCTCAGCGAGT (SEQ ID NO:91) carbon spacer TCTCGGATCCTCAGCGAGT 3′ (SEQ ID NO:91)). RNA aptamers to be dimerized were mixed with this linker at a 2:1 molar ratio of RNA to linker. The mixture was heated to 95° C. for 5 minutes followed by slow cooling to room temperature, resulting in dimers being formed as a result of hybridization formed during this annealing process. For binding affinity determination, aptamer dimers were purified using 8% native PAGE purification, followed by overnight elution into physiological buffer at 4° C., and followed by extensive washing. For binding affinity determinations, purified RNA dimers were 3′ radiolabelled by incubation with T4 RNA ligase according to manufacturer's instructions at 4° C. Binding affinities were determined for the aptamer monomer, and the aptamer dimers, using the filter-binding assay as previously described herein.

### Methods for Identifying an Agonist Aptamer

A method for identifying a nucleic acid aptamer having agonistic activity comprises:
(a) contacting a nucleic acid aptamer or multimer thereof with cells comprising a ligand for which the nucleic acid aptamer has binding affinity; and
(b) detecting a cell activity (e.g., one or more cell activities), which activity is initiated by cell signaling resulting from the binding of the nucleic acid aptamer to the ligand of which the cells are comprised;
   wherein detection of the cell activity is an indication that the nucleic acid aptamer comprises an agonist aptamer.

In this illustrative example of the method according to the invention, nucleic acid aptamers having binding affinity for OX40 (CD134) are contacted with cells expressing OX40 as a ligand of the cells and which is exposed at the cell surface, and then the cells are assayed for a cell activity comprising cell proliferation. The effect of the dimerized aptamers on activation of ligand OX40 was tested, as was the consequent increase in lymph node cell proliferation. 50 μg of the antigen Staphyloccocal enterotoxin B (“SEB”) resuspended in physiological buffer was administered to female Balb/c mice intraperitoneally. SEB antigen was used as a co-stimulatory signal to activate T cells. Auxiliary, inguinal and mesenteric lymph nodes were harvested after 24 hours. Cells were teased into single cell suspensions, and then labelled with carboxyfluoroscein succinimidyl ester (CFSE) by incubating cells at a concentration of 1 million cells/mL in buffer containing 5% fetal bovine serum and 2 mM CFSE at room temperature for 5 minutes. Cells were washed twice using phosphate buffered saline with 5% fetal bovine serum, followed by a final wash with tissue culture medium containing 10% fetal bovine serum. 10⁵ cells were seeded in wells of a round bottom 96 well plate, and were cultured (in a humidified chamber at 37° C./5% CO₂) for 72 hours in tissue culture medium containing 10% fetal bovine serum in the presence of 0.5 ng/mL Staphyloccocal enterotoxin and one of: OX40 agonistic antibody (OX86, 33 nM) as a positive control; isotype control antibody (33 nM) as an assay negative control; aptamer dimer having binding affinity for OX40 (66 nM; see e.g., Table 2); or aptamer dimer lacking detectable binding to OX40 (66 nM) as an assay negative control. Cell proliferation data was collected using flow cytometry, and evaluated using commercially available software. Treatment of the co-stimulated T cells with either the aptamer dimer having binding affinity for OX40 (e.g., having a variable region with a nucleotide sequence comprising SEQ ID NO:77) or an agonistic anti-OX40 antibody led to an increase in T cell proliferation. The percent proliferation induced by the agonistic anti-OX40 antibody is not significantly different from the percentage induced by OX40 activation using the dimerized aptamer (p>0.05). However, the increase in proliferation engendered by the dimerized aptamer and agonistic antibody is statistically significant compared to the respective assay negative control (p<0.05).

In another illustrative example of the method according to the invention, the nucleic acid aptamer is contacted with cells, and then the cells are assayed for a cell activity comprising cytokine production (e.g., by detecting cytokines released from the cell treated with the nucleic acid aptamer). It is known that activation of OX40 receptor on cells results in interferon-gamma secretion by cells. Supernatants of proliferation assay replicates (as described above) were pooled after 72 hours of culture. Interferon-gamma secretion was measured in triplicate using a commercially available ELISA kit following the manufacturer's instructions. The agonistic OX40 antibody led to a significant induction of interferon-gamma secretion (e.g., about 15 pg/mL after subtracting out the background observed with the respective assay negative control). Surprisingly, the aptamer dimer having binding affinity for OX40 induced even a greater amount of (about 2 fold more) interferon-gamma production (e.g., about 30 pg/mL after subtracting out the background observed with the respective assay negative control) than the agonistic anti-OX40 antibody. Another cell activity associated with activation and signaling via the OX40 receptor is nuclear localization of NF kappaB. Western blot analysis, performed on nuclear fractions isolated from treated T cells as described above, confirmed that treatment with either aptamer dimer having binding affinity for OX40 or agonistic anti-OX40 antibody, but not the respective negative controls, resulted in nuclear localization of NF kappaB. It is important to note that in demonstrating agonistic activity of multimerized RNA aptamers having binding affinity for OX40 in this assay and the other assays described above, RNA molecules (lacking detectable binding affinity for OX40) comprising dimers were used as assay negative controls. Thus, the results of these assays confirm that the activity of the nucleic acid aptamer dimers in stimulating a cell activity is a result of agonist activity (i.e., mediated by agonist aptamer binding its ligand) rather than by nonspecific effects on the cells mediated by the molecules being RNA in nature.

To determine if agonist aptamer binding to OX40 can also act as an agonist in vivo, the aptamer's ability to induce OX40 function was evaluated in a tumor immunotherapy setting. More precisely, an agonist aptamer dimer described in this Example 5 was evaluated for the ability to enhance antitumor responses generated by dendritic cells (DC) transfected with tumor antigen. Female C57/BL6 mice were implanted with B16-F10.9 melanoma tumor cells and vaccinated with DCs pulsed with either the melanoma antigen tyrosinase-related protein 2 (TRP-2) or actin (negative control) mRNA. This vaccine was administered in the presence of either agonistic aptamer dimer having binding affinity for OX40, agonistic anti-OX40 antibody, or the respective assay negative controls (as described above). As shown in FIG. 3, administration of DCs containing the TRP-2 antigen alone (i.e., administered with the either of the respective assay negative controls; line with open circles represents DC/Trp2 with isotype control antibody, and line with open squares represents DC/Trp2 with control RNA aptamer) delayed the development of a palpable tumor, as compared to assay negative control antigen (DC transfected with actin)-treated animals. However such treatment with DC/Trp2 did not lead to a cure in mice. Rather, administration of either an agonistic aptamer dimer having binding affinity for OX40 (FIG. 3, line with closed squares; Dc/TRP-2+OX40 aptamer) or an agonistic anti-OX40 antibody (FIG. 3, line with closed circles; Dc/TRP-2+OX40 Ab) to animals receiving the DC-TRP-2 vaccination resulted in tumor eradication in 30-40% of the animals (p≦0.05). Therefore, an agonistic aptamer dimer having binding affinity for OX40 can mediate tumor regression in comprising a potent adjuvant for a DC-based tumor vaccine in vivo.

Thus, again demonstrated are (a) a method for identifying an agonist aptamer; (b) agonist aptamers, and particularly aptamers comprising multimers which can function as agonists; (c) a ligand which can be bound by agonist aptamer; and (d) examples of cell activities which can be used to detect agonist aptamer function or identification.

### Example 6

The previous examples show that despite the compositional, functional, size (e.g., on average, an aptamer being about one fifth to one sixth the size of an antibody molecule), and structural differences between nucleic acid aptamers and antibodies, surprisingly and unexpectedly, nucleic acid aptamers can function as agonists of ligands that bind with the nucleic acid aptamers. Demonstrated are agonist aptamers comprising nucleic acid aptamers of from about 20 to about 80 nucleotides (optionally, comprising modified nucleic acid bases), which are then oligomerized (multimerized) such as in dimer form, and which bind to a ligand for the agonist expressed by cells such that induced is cell signaling that triggers one or more biological activities of the cells. Demonstrated is the use of a linker to multimerize aptamers in forming agonist aptamers, wherein the linker comprised a carbon linker, a nucleic acid molecule linker, or a combination thereof. Typically, the linker comprised a length of from about 3 nm to about 20 nm, and more preferably, from about 5 nm to about 10 nm. Also, demonstrated were illustrative examples of ligands comprising molecules or receptors expressed on a cell surface and/or as a component of the cell membrane which, when bound and crosslinked by agonist aptamer, resulted in signal transduction that triggers cell activity. Additionally, demonstrated were illustrative examples of cell activity triggered as a result of agonist aptamer-ligand binding interactions (e.g., crosslinking), such as induction of one or more of cytokine production, apoptosis, cell proliferation, cell differentiation, cell survival, an immune response, reversal of cell anergy.

The previous examples also show illustrative examples of a method according to the present invention for successfully identifying a nucleic acid aptamer having agonistic activity, i.e., an agonist aptamer; and, after identification of an agonist aptamer, a method for producing agonist aptamer (i.e., synthesizing agonist aptamer). An agonist aptamer according to the invention can be readily produced in large quantities by one or more standard means known in the art for nucleotide synthesis including, but not limited to, chemical synthesis, enzymatic synthesis, recombinant synthesis, and chemical or enzymatic cleavage from a larger precursor nucleic acid molecule. For example, described herein is use of chemical or enzymatic synthesis of RNA aptamers using an RNA polymerase for transcription of a DNA template. Synthesis may be in vitro, in vivo, automated, manual, or a combination thereof. In producing an agonist aptamer, the agonist aptamer may be purified from other components used in the synthetic process to result in a preparation comprising isolated agonist aptamer. Deprotection, purification, and analytic methods for nucleic acid molecule synthesis are well known in the art.

One or more modified nucleic acid bases (as described previously in more detail herein) may be incorporated during the synthesis of an agonist aptamer molecule such that the agonist function is not substantially affected. For example, agonist aptamers produced in accordance with the invention were synthesized to incorporate modified nuclei acid bases comprising 2′OH purines and 2′F pyrimidines. The amount and location of incorporation of modified nucleic acid bases into an agonist aptamer can be monitored for any effect on agonist activity by screening such nucleic acid aptamers for retention of agonistic function, such as by the methods described herein for identifying an agonist aptamer. In some cases, an agonist aptamer comprising modified nucleic acid bases may display one or more improved properties as compared to an agonist aptamer containing only naturally occurring and/or unmodified nucleotides. Such properties may include, but are not limited to, reduced digestion by exonucleases, improved stability, and the like.

Additionally, the agonist aptamer may be directly synthesized as a multimer (e.g., dimer). For example, in synthesis as one molecule (e.g., by linear synthesis), a monomer of the nucleic acid aptamer is synthesized, followed by synthesis of a nucleic acid molecule linker, followed by synthesis of another monomer of the nucleic acid aptamer in forming a dimer agonist aptamer. Alternatively, two or more monomers of a nucleic acid aptamer may be separately synthesized, and then linked together by base pairing with a nucleic acid molecule sharing complementarity, as previously described and shown herein in more detail. As another alternative, two or more monomers may be operatively linked to the respective ends of a linker using chemical means such as covalent bond formation, by methods known to those skilled in the art. For example, modified nucleic acid bases may contain a functional chemical moiety (e.g., OH, H, OR, R, halo, SH, NH2, or CN) which may be used to chemically bond to a corresponding reactive chemical moiety of the linker using methods known in the art.

Further, as for example described above for RNA molecules, agonist aptamers according to the invention may further comprise a functional and smaller agonist aptamer produced from an agonist aptamer identified according to the method of the invention. For example, using methods known in the art (e.g., in performing polymerase chain reaction), deletions or truncations may be made of the originally identified agonist aptamer, such as by systematically deleting portions of the constant region, that can result in an agonist aptamer still retaining its biologically relevant shape and agonistic function. Retention of agonistic function can be evaluated by the methods described herein for identifying an agonist aptamer.

An agonist aptamer according to the invention may be administered once, or multiple times, as needed, to deliver an amount of the agonist aptamer effective to mediate agonistic activity upon binding to cells comprising the ligand to which binds agonist aptamer. If co-stimulation is needed for optimal agonistic activity of an agonist aptamer according to the invention, administration of the agonist aptamer to a cell or individual may optionally or further comprise administration of an agonist antibody, or another agonist aptamer, having agonistic function for the co-stimulatory receptor (“co-stimulatory agonist”). For example, in the Examples herein, demonstrated was co-stimulation of cells with a co-stimulatory agonist comprising an anti-T cell antibody, followed by administration of agonist aptamer. The co-stimulatory agonist and agonist aptamer according to the invention may be jointly or separately administered, in either order, substantially proximate (e.g., within minutes or hours) or simultaneous to one another, in an amount and under conditions that result in the desired co-stimulation and agonistic function (“effective amount”). A method for determining an effective amount may comprise, but is not limited to, monitoring for one or more cell activities triggerable by agonist aptamer binding to ligand, following administration of the agonist aptamer. An effective amount of the one or more agonistic compositions will depend on such factors as the mode of administration, the formulation for administration, the cell activity to be induced, the size and health of the individual to receive such a composition, and other factors which can be taken into consideration by a medical practitioner whom is skilled in the art of determining appropriate dosages for treatment. An amount to be administered may vary from 0.00001 grams to about 5 grams, and more typically from about 0.001 grams to about 1 gram. An agonist aptamer may further comprise a pharmaceutically acceptable carrier, such as for facilitating one or more of storage, stability, administration, and delivery, of the agonist aptamer. Thus, provided is a pharmaceutical composition comprising an agonist aptamer and a pharmaceutically acceptable carrier. The mode of administration may be any mode known in the art to be suitable for delivering the agonist aptamer to the cells to be activated.

The foregoing description of the specific embodiments of the present invention have been described in detail for purposes of illustration. In view of the descriptions and illustrations, others skilled in the art can, by applying, current knowledge, readily modify and/or adapt the present invention for various applications without departing from the basic concept of the present invention; and thus, such modifications and/or adaptations are intended to be within the meaning and scope of the appended claims.

All documents and other information sources cited above, and/or in PCT/US2007/022357 and/or in U.S. application Ser. No. 12/066,598, are hereby incorporated in their entirety by reference.

1. A method of identifying a nucleic acid aptamer having agonistic activity (“agonist aptamer”) comprising:
(a) contacting a nucleic acid aptamer or multimer thereof with cells comprising a ligand to which the nucleic acid aptamer binds; and
(b) detecting a cell activity, which activity is initiated by cell signaling resulting from the binding of the nucleic acid aptamer to the ligand;
wherein detection of the cell activity is an indication that the nucleic acid aptamer comprises an agonist aptamer.

2. An agonist aptamer comprising a nucleic acid aptamer made by
(a) contacting the nucleic acid aptamer, or multimer thereof, with cells comprising a ligand to which the nucleic acid aptamer binds; and
(b) detecting a cell activity, which activity is initiated by a cell signal resulting from the binding of the nucleic acid aptamer to the ligand, the cell activity comprising induction of one or more of cytokine production, apoptosis, cell proliferation, cell differentiation, cell survival, an immune response, and reversal of cell anergy; and
wherein detection of the cell activity is an indication that the nucleic acid aptamer comprises an agonist aptamer.

3. The agonist aptamer of claim 2, wherein the ligand comprises a cell surface molecule comprising a glycosyiphosphatidylinositil (GPI)-anchored molecule, or a receptor of the Tumor Necrosis Factor (TNF) superfamily of receptors, or insulin receptor; and wherein the agonist aptamer comprises a multimer comprising two or more aptamer molecules linked together by a linker.

4. The agonist aptamer of claim 2, wherein the agonist aptamer comprises a dimer.

5. The agonist aptamer of claim 3, wherein the agonist aptamer comprises a dimer.

6. A pharmaceutical composition comprising agonist aptamer according to claim 2, and a pharmaceutically acceptable carrier.

7. A pharmaceutical composition comprising agonist aptamer according to claim 3, and a pharmaceutically acceptable carrier.

8. The pharmaceutical composition of claim 3, wherein agonist aptamer comprises an agonist aptamer that binds to a ligand CD134, and an agonist aptamer that binds to a ligand CD137.

9. The pharmaceutical composition of claim 8, wherein the agonist aptamer, that binds to a ligand CD134, comprises a dimer; and the agonist aptamer, that binds to a ligand CD137, comprises a dimer.

10. An isolated nucleic acid aptamer comprising a multimer of at least two molecules of the nucleic acid aptamer joined by a linker comprising a carbon linker; wherein the nucleic acid aptamer comprises agonist activity mediated by binding and crosslinking a ligand on cells comprising a ligand for the agonist aptamer.

11. A method of producing a nucleic acid aptamer having agonistic activity (“agonist aptamer”) comprising:
(a) contacting a nucleic acid aptamer, or multimer thereof, with cells comprising a ligand to which the nucleic acid aptamer binds; wherein the ligand comprises a cell surface molecule expressed by cells comprising T cells; and wherein crosslinking of the ligand by the nucleic acid aptamer results in cell signaling; and
(b) detecting a cell activity, which activity is initiated directly or indirectly by cell signaling resulting from the binding of the nucleic acid aptamer to the ligand;
wherein detection of the cell activity is an indication that the nucleic acid aptamer comprises an agonist aptamer; and
(c) synthesizing the agonist aptamer, in producing agonist aptamer.

12. An agonist aptamer comprising a nucleic acid aptamer made by
(a) contacting the nucleic acid aptamer, or multimer thereof, with cells comprising a ligand to which the nucleic acid aptamer binds; wherein the ligand comprises a cell surface molecule expressed by cells comprising T cells; and wherein crosslinking of the ligand by the nucleic acid aptamer results in cell signaling; and
(b) detecting a cell activity, which activity is initiated by a cell signal resulting from the binding of the nucleic acid aptamer to the ligand, the cell activity comprising induction of one or more of cytokine production, apoptosis, cell proliferation, cell differentiation, cell survival, an immune response, and reversal of cell anergy; and
wherein detection of the cell activity is an indication that the nucleic acid aptamer comprises an agonist aptamer.

13. The method of claim 11, wherein the ligand comprises a glycosylphosphatidylinositil (GPI)-anchored molecule, or a receptor of the Tumor Necrosis Factor (TNF) superfamily of receptors, or insulin receptor.

14. The agonist aptamer of claim 12, wherein the ligand comprises a glycosylphosphatidylinositil (GPI)-anchored molecule, or a receptor of the Tumor Necrosis Factor (TNF) superfamily of receptors, or insulin receptor.

15. A method of inducing a biological activity by cells, the method comprising contacting the cells, having a cell surface ligand capable of binding agonist aptamer according to claim 2, with an effective amount of agonist aptamer to bind the ligand, wherein binding of agonist aptamer to the ligand results in cell signaling that induces the biological activity by the cells.


SEQUENCE LISTING (91 sequences)

SEQ ID NO: 1 - RNA, 40 nt, Artificial
  synthesized
cgaccgaacgugcccuucaaagccguucacuaaccagugc

SEQ ID NO: 2 - RNA, 40 nt, Artificial
  synthesized
cgaccgaacgugcccuucaaagccguucacuaaccagugg

SEQ ID NO: 3 - RNA, 40 nt, Artificial
  synthesized
cgaccgaacgugcccuucaaagccguucacuaaccaguga

SEQ ID NO: 4 - RNA, 40 nt, Artificial
  synthesized
gaagugacagcucccagcgcuucaaagcucaucuauaacu

SEQ ID NO: 5 - RNA, 40 nt, Artificial
  synthesized
cagaaacuagaccuccgaucggacacccggucccuucguc

SEQ ID NO: 6 - RNA, 40 nt, Artificial
  synthesized
gaagcacaauaggccgcaacacuucaaaacccauucaauc

SEQ ID NO: 7 - RNA, 39 nt, Artificial
  synthesized
caagcacucuucaggcuaaggacucucuugacaccccgc

SEQ ID NO: 8 - RNA, 40 nt, Artificial
  synthesized
gcacagcaacaccacgacccccccuaggcuuccgcccgcc

SEQ ID NO: 9 - RNA, 40 nt, Artificial
  synthesized
gcacagcaacaccacgacccccccuaggcuuccgcccgcg

SEQ ID NO: 10 - RNA, 40 nt, Artificial
  synthesized
gcacagcaacaccacgacccccccuaggcuuccgcccgca

SEQ ID NO: 11 - RNA, 41 nt, Artificial
  synthesized
uaacggcccaaugacuucgccuuacugccccccuaagcuuc

SEQ ID NO: 12 - RNA, 40 nt, Artificial
  synthesized
aaagcgacaauucuuacuacuccccaagcuccacgccuuu

SEQ ID NO: 13 - RNA, 40 nt, Artificial
  synthesized
aagacgauaccuagccucaaaauuccucccccgacuuccu

SEQ ID NO: 14 - RNA, 40 nt, Artificial
  synthesized
cgagaacccgcaucuucggaugcgccccccuaggacuuac

SEQ ID NO: 15 - RNA, 40 nt, Artificial
  synthesized
gaccaagggcagcaucaccguucccccccuaggagcuuac

SEQ ID NO: 16 - RNA, 39 nt, Artificial
  synthesized
aacggcccaaugacuucgccuucugccccccuaagcuuc

SEQ ID NO: 17 - RNA, 40 nt, Artificial
  synthesized
cgcucucucacaaccacgaccuccgaucugauaauucguc

SEQ ID NO: 18 - RNA, 40 nt, Artificial
  synthesized
gcaccaaacaccgguucagaacccaucauguaacuccuug

SEQ ID NO: 19 - RNA, 40 nt, Artificial
  synthesized
aacuaccuccucgaaccauaguucaacaccauccagccau

SEQ ID NO: 20 - RNA, 40 nt, Artificial
  synthesized
cuccuagacagacgaugucugaacgacucuugaagggcag

SEQ ID NO: 21 - RNA, 40 nt, Artificial
  synthesized
ccgccauuaacccugaacagaaucuuccuaucccaacccg

SEQ ID NO: 22 - RNA, 40 nt, Artificial
  synthesized
ucacccgacgauaccuaacuuccuccgacaagccuuugcc

SEQ ID NO: 23 - RNA, 40 nt, Artificial
  synthesized
gcacaacauccucnguganaccacacuucgccaancucug

SEQ ID NO: 24 - RNA, 39 nt, Artificial
  synthesized
aucaguacaugcuccggggcagccgaauaucucgcgcuu

SEQ ID NO: 25 - RNA, 39 nt, Artificial
  synthesized
gaccaccacagcgacaggcucgcacgacucucgaagagg

SEQ ID NO: 26 - RNA, 39 nt, Artificial; identical to SEQ ID NO: 7
  synthesized

SEQ ID NO: 27 - RNA, 40 nt, Artificial
  synthesized
ccuccuuccuaacccgcaaacacgauuggcucgaaacuuc

SEQ ID NO: 28 - RNA, 39 nt, Artificial
  synthesized
aaccgaccgcagaucuggacacuagcuuucgcgaggauu

SEQ ID NO: 29 - RNA, 40 nt, Artificial
  synthesized
gcuucuacaggggguaaagauucgcucaucacucgcaaaa

SEQ ID NO: 30 - RNA, 40 nt, Artificial; identical to SEQ ID NO: 2
  synthesized

SEQ ID NO: 31 - RNA, 40 nt, Artificial
  synthesized
gcacucuuacuucucgcccaccaaccgaacccagccaucu

SEQ ID NO: 32 - RNA, 40 nt, Artificial
  synthesized
cugacugccacuucuucccagcaaucuucaccguccuucu

SEQ ID NO: 33 - RNA, 40 nt, Artificial
  synthesized
aaguuaaucgcgccuuaccagcuucaggaccauucaauuc

SEQ ID NO: 34 - RNA, 40 nt, Artificial
  synthesized
cuuccacacucuccuagcuggacaacagacuaaccuucuu

SEQ ID NO: 35 - RNA, 39 nt, Artificial
  synthesized
cacccgucugccagacgcucgacccaaacuccagccucu

SEQ ID NO: 36 - RNA, 40 nt, Artificial
  synthesized
ugauccaaaaccaaauagcaguagacucucgaagaggacg

SEQ ID NO: 37 - RNA, 40 nt, Artificial
  synthesized
caaacccaugcaagacucuucaaucuccaaccccuuccaa

SEQ ID NO: 38 - RNA, 40 nt, Artificial
  synthesized
cuucaacaacgacucgccauacaacaacacacaaccccuu

SEQ ID NO: 39 - RNA, 40 nt, Artificial
  synthesized
gaagaccaaacgagccaccuucaagucccauccaaccccu

SEQ ID NO: 40 - RNA, 40 nt, Artificial
  synthesized
cgucaaccccuuucagucuccccguaagcaucucuuccac

SEQ ID NO: 41 - RNA, 40 nt, Artificial
  synthesized
cuccaauccccgaaaucuucaagcugcucuccagucccca

SEQ ID NO: 42 - RNA, 39 nt, Artificial
  synthesized
acacugaaaacgggcaagugacuacgauucgugucagac

SEQ ID NO: 43 - RNA, 40 nt, Artificial
  synthesized
cuccuagucagacgaugucugaacgacucuugaagggcag

SEQ ID NO: 44 - RNA, 40 nt, Artificial
  synthesized
uaagcggccucgaacaccgauugacuacugcccgaacauc

SEQ ID NO: 45 - RNA, 40 nt, Artificial
  synthesized
cccccucgcaagacccucaucgcaucucaagacaccuacc

SEQ ID NO: 46 - RNA, 40 nt, Artificial
  synthesized
acgaccaccaacgauugagcagcuagauucgaccgacacu

SEQ ID NO: 47 - RNA, 40 nt, Artificial
  synthesized
ugcgauccacggucuauucaccaguaaccucugaccaaaa

SEQ ID NO: 48 - RNA, 40 nt, Artificial
  synthesized
cuccagacauaguauccgccaguaaccucuguaacgcccg

SEQ ID NO: 49 - RNA, 40 nt, Artificial
  synthesized
cgcacuaccugcacauugaccaguaccccugugccacgcu

SEQ ID NO: 50 - RNA, 40 nt, Artificial
  synthesized
ccgacgaccuccuucaccuccccuuccucgaacuccaacc

SEQ ID NO: 51 - RNA, 40 nt, Artificial
  synthesized
accugacgcauaccuccucccaccgcaagugcccuccgcu

SEQ ID NO: 52 - RNA, 40 nt, Artificial
  synthesized
acgaccucuuugauuaccugggcugauuugccaucuucac

SEQ ID NO: 53 - RNA, 40 nt, Artificial
  synthesized
acgaccucuuugauuaccugggcugauucgccaucuucac

SEQ ID NO: 54 - RNA, 40 nt, Artificial
  synthesized
ccucacugcguaauccgccgugauucaccgcuguacacga

SEQ ID NO: 55 - RNA, 40 nt, Artificial
  synthesized
caccaucgauaucacgcacugacaccaguacccuugcgca

SEQ ID NO: 56 - RNA, 40 nt, Artificial
  synthesized
ugcuccaaucggaaccaucucgccgcucgcucuccauuac

SEQ ID NO: 57 - RNA, 40 nt, Artificial
  synthesized
gaaaccugugcgcaugaagagcugcuaacccguuacgaac

SEQ ID NO: 58 - RNA, 40 nt, Artificial
  synthesized
gaucaaacuaaucgcccucuaacuugaucggcccgacucc

SEQ ID NO: 59 - RNA, 40 nt, Artificial
  synthesized
cccccgcguccucccaacccaaaauuccccuggcacccuc

SEQ ID NO: 60 - RNA, 40 nt, Artificial
  synthesized
ugcucuuaaucucccuaagaaccaucuccauaguccuccg

SEQ ID NO: 61 - RNA, 39 nt, Artificial
  synthesized
ggaucgacccgcuugcugcgacacgaccgucguaaccaa

SEQ ID NO: 62 - RNA, 40 nt, Artificial
  synthesized
uggaucucgcucuggcgcuuagcuccgcaaaucugccguu

SEQ ID NO: 63 - RNA, 41 nt, Artificial
  synthesized
cacuuccacucacaacggcaaaacuucaacuccaauacccu

SEQ ID NO: 64 - RNA, 40 nt, Artificial
  synthesized
accagcuucccugggccugcugugaacuccagccugacua

SEQ ID NO: 65 - RNA, 40 nt, Artificial
  synthesized
gccuuuacaccgacccugccaagaacgaagcgcgaacaac

SEQ ID NO: 66 - RNA, 40 nt, Artificial
  synthesized
caccuucccuuucuccaagacaagccucaugucaaacucu

SEQ ID NO: 67 - RNA, 40 nt, Artificial
  synthesized
ggcuucccugcacuugaucuggcccgaccaccagccugug

SEQ ID NO: 68 - RNA, 40 nt, Artificial
  synthesized
cgauccucuguacacgacacucauuccacaguacgggacg

SEQ ID NO: 69 - RNA, 40 nt, Artificial
  synthesized
cacccuucgucucaaccacccaacucuccaugucccuaac

SEQ ID NO: 70 - RNA, 40 nt, Artificial
  synthesized
caaacuucccugugguuauagcacugcguugggucccuug

SEQ ID NO: 71 - RNA, 40 nt, Artificial
  synthesized
auaccuguaccuugcaacacugcccgacgacgcacccgca

SEQ ID NO: 72 - RNA, 40 nt, Artificial
  synthesized
ccacacgcuacuaucaaccgcugcguucaaacuugggauc

SEQ ID NO: 73 - RNA, 40 nt, Artificial
  synthesized
gaccagucuaaccauccacacucugucauugcccagcgcc

SEQ ID NO: 74 - RNA, 40 nt, Artificial
  synthesized
accuuuaccgccgacccggacagagugagccucugcagag

SEQ ID NO: 75 - RNA, 40 nt, Artificial
  synthesized
cuccagaacccuucgaccugcacgacggucuuccucgcgu

SEQ ID NO: 76 - RNA, 96 nt, Artificial
  synthesized
gggagagaggaagagggaugggnnnnnnnnnnnnnnnnnnnnnnnnnnnnnnnnnnnnnnnncauaacccagaggucgauaguacuggaucccccc

SEQ ID NO: 77 - RNA, 40 nt, Artificial
  synthesized
cagucugcaucguaggaaucgccaccguauacuuucccac

SEQ ID NO: 78 - RNA, 40 nt, Artificial
  synthesized
auaccaggaucacauccugaggaaccccggcucccaaccu

SEQ ID NO: 79 - RNA, 40 nt, Artificial
  synthesized
cuuuaauccucgcacucagcgcgcaucacccuugacauca

SEQ ID NO: 80 - RNA, 39 nt, Artificial
  synthesized
caaaccagcuauuuccugagguaccccggcucuccaugg

SEQ ID NO: 81 - RNA, 39 nt, Artificial
  synthesized
auaccagcgaauaacucgcugaggaacccgacucacaaa

SEQ ID NO: 82 - RNA, 40 nt, Artificial
  synthesized
cuuuugaaugcucccuucacuccaagcggcagccuaucgu

SEQ ID NO: 83 - RNA, 39 nt, Artificial
  synthesized
auaccaccuagugugaggaaccccgcuuccuagacugcg

SEQ ID NO: 84 - RNA, 40 nt, Artificial
  synthesized
caacccauccauaccaacauggagcuaauguuagucaacg

SEQ ID NO: 85 - RNA, 40 nt, Artificial
  synthesized
cacuaacuccagcgacuccaucgccauuccaacucuacgc

SEQ ID NO: 86 - RNA, 40 nt, Artificial
  synthesized
ccgucaaccaaucucucgcuucacacggccaugccuccau

SEQ ID NO: 87 - RNA, 106 nt, Artificial
  synthesized
ggggaauucuaauacgacucacuauagggaggacgaugcggnnnnnnnnnnnnnnnnnnnnnnnnnnnnnnnnnnnnnnnncagacgacucgcugaggauccgaga

SEQ ID NO: 88 - RNA, 96 nt, Artificial
  synthesized
gggagagaggaagagggaugggcgaccgaacgugcccuucaaagccguucacuaaccagugacauaacccagaggucgauaguacuggaucccccc

SEQ ID NO: 89 - RNA, 117 nt, Artificial
  synthesized
gggagagaggaagagggaugggcgaccgaacgugcccuucaaagccguucacuaaccagugacauaacccagaggucgauaguacuggauccccccgcuauaagugugcaugagaac

SEQ ID NO: 90 - RNA, 117 nt, Artificial
  synthesized
gggagagaggaagagggaugggcgaccgaacgugcccuucaaagccguucacuaaccagugacauaacccagaggucgauaguacuggauccccccguucucaugcacacuuauagc

SEQ ID NO: 91 - DNA, 19 bp, Artificial Sequence
  synthesized
tctcggatcctcagcgagt